(19) United States
(12) Patent Application Publication
(10) Pub. No.: US 2022/0241758 A1
(43) Pub. Date: Aug. 4, 2022

(54) SELECTIVE PRODUCTION OF METHANOL AND ETHANOL FROM CO HYDROGENATION

(71) Applicant: SABIC Global Technologies B.V., Bergen op Zoom (NL)

(72) Inventors: Muhammad H. HAIDER, Riyadh (SA); Waleed K. AL-DAHLOUS, Riyadh (SA)

(73) Assignee: SABIC Global Technologies B.V., Bergen op Zoom (NL)

(21) Appl. No.: 17/626,889

(22) PCT Filed: Jul. 9, 2020
(86) PCT No.: PCT/IB2020/056474

§ 371 Date: Jan. 13, 2022

Related U.S. Application Data
(60) Provisional application No. 62/875,472, filed on Jul. 17, 2019.

(51) Int. Cl. B01J 23/882; B01J 37/03; B01J 37/04; B01J 37/08; B01J 37/18; C07C 29/156

(52) U.S. Cl. CPC B01J 23/882; B01J 37/031; C07C 29/156; B01J 37/082; B01J 37/18; B01J 37/04

(57) ABSTRACT

A method for producing methanol and ethanol is disclosed. The method can include contacting a gaseous stream comprising carbon monoxide (CO) and hydrogen (H₂) with a crystalline cobalt molybdenum catalyst under conditions suitable to produce a products stream comprising methanol and ethanol from the CO and H₂.

### CROSS REFERENCE TO RELATED APPLICATIONS

This application claims benefit of priority of U.S. Provisional Patent Application No. 62/875,472 filed Jul. 17, 2019, which is hereby incorporated by reference in its entirety.

### FIELD OF INVENTION

The invention generally concerns compositions and methods for selective production of methanol and ethanol. In particular, the invention concerns the use of a crystalline cobalt (Co) molybdenum (Mo) catalyst for selective production of methanol and ethanol from a mixture of carbon monoxide (CO) and hydrogen (H₂).

### BACKGROUND OF THE INVENTION

Methanol and ethanol are important chemicals with multiple industrial uses. For example, methanol is used as fuels, as feed stock for plastic industry, and in many other processes. Ethylene, a dehydration product of ethanol, is an important raw material for multiple end products like polymers, rubbers, plastics etc. It is expected that demand for methanol and ethanol will continue to grow.

However, there is an absence of suitable catalysts that can produce methanol and ethanol with high selectivity. By way of example, U.S. Pat. No. 9,409,840 discloses the use of a carbon supported Co/Mo catalyst for producing C2-C3 alcohols, with selectivity of C3 alcohols being relatively high at least 19% and up to 33%.

### SUMMARY OF THE INVENTION

A discovery has been made that provides a solution to at least some of the aforementioned problems associated with the production of methanol and ethanol. The solution is premised on the use of a crystalline cobalt molybdenum catalyst to catalyze the production of methanol and ethanol from a mixture of carbon monoxide (CO) and hydrogen (H₂). In particular, it has been discovered that the crystal structure of the catalyst provides for a relatively high selectivity for methanol and ethanol production via CO hydrogenation. By way of example, and as illustrated in a non-limiting manner in the Examples, the combined selectivity of methanol and ethanol can be at least 50%, and the selectivity of propanol can be less than 10%. Further the individual selectivity for methanol and ethanol can each be at least 30%. In some specific aspects, the crystalline cobalt molybdenum catalyst can include a monoclinic crystalline system.

In one aspect of the present invention, a method to produce methanol and ethanol is described. The method can include a step of contacting a gaseous stream containing CO and H₂ with a crystalline cobalt molybdenum catalyst under conditions suitable to produce a products stream containing methanol and ethanol from the CO and H₂. The conditions can include a pressure of 25 to 90 bar, a GHSV of 1000 to 3000 h⁻¹, and/or a temperature of 150 to 450° C. In some aspects, the CO conversion can be 20% to 40%, preferably 25% to 40%, more preferably 28% to 40%. In some aspects, the combined selectivity of the methanol and ethanol produced from the CO and H₂ can be 50% to 75%. In some particular aspects, selectivity of the methanol can be 20% to 40%, preferably 25% to 40%, more preferably 30% to 35%. In some particular aspects, selectivity of the ethanol can be 20% to 40%, preferably 25% to 40%, more preferably 30% to 35%. In some aspects, combined selectivity of propanol and butanol if produced from the CO and H₂ can be less than 20%, preferably less than 15%, more preferably less than 10%. In some particular aspects, selectivity of propanol if produced can be less than 10%, preferably less than 7%, more preferably less than 5%. In some particular aspects, selectivity of butanol if produced can be less than 10%, preferably less than 7%, more preferably less than 5%. The molar ratio of H₂ and CO in the gaseous stream can be in the range 0.5:1 to 3:1, preferably 0.8:1 to 1.2:1. In some aspects, the crystalline cobalt molybdenum catalyst can include a monoclinic cobalt molybdenum catalyst. A monoclinic cobalt molybdenum catalyst can have a monoclinic crystalline system (monoclinic crystalline system or structure can be used interchangeably in this specification). In some aspects, the monoclinic cobalt molybdenum catalyst can be a monoclinic cobalt molybdenum oxide. In some aspects, the monoclinic cobalt molybdenum oxide can be CoₓMo_{y}O_{z}, with x ranging from 0.5 to 1.5, preferably 0.9 to 1.1, y ranging from 0.5 to 1.5, preferably 0.9 to 1.1, and z can be a value that balances the valencies of Co and Mo. In certain aspects, z can be 3.5 to 4.5, preferably 3.9 to 4.1. In some particular aspects, the monoclinic cobalt molybdenum oxide can contain α-CoMoO4 and β-CoMoO₄ and the wt. % ratio of α-CoMoO₄ to β-CoMoO₄ can be 15:85 to 35:65, preferably 20:80 to 30:70. In some aspects, the crystalline cobalt molybdenum catalyst can be activated, prior to contacting the catalyst with the gaseous stream. The catalyst can be activated by reduction with hydrogen (H₂). In certain instances, the activation process can include reducing the catalyst with a stream containing hydrogen (H₂) at a temperature 200° C. to 500° C., at a GHSV of 1000 to 3000 , and/or at pressure 25 bar to 90 bar for 8 h to 20 h. The catalyst can be a bulk catalyst or a supported catalyst, preferably a bulk catalyst.

In one aspect of the present invention a method for preparing a crystalline cobalt molybdenum catalyst is described. The method can include preparing a solution containing a cobalt compound and a molybdenum compound, collecting a precipitate from the solution, drying the precipitate to obtain a dried precipitate, and calcining the dried precipitate to obtain the crystalline cobalt molybdenum catalyst. The solution can be an aqueous solution. The cobalt compound can be cobalt acetate, cobalt acetyl acetonate, cobalt citrate, or a combination thereof, preferably cobalt acetate. The molybdenum compound can be ammonium heptamolybdate, molybidic acid, phosphomolybdic acid, potassium heptamolybdate, or a combination thereof, preferably ammonium heptamolybdate. The precipitate can be dried at a temperature of 70° C. to 150° C. for 3 h to 10 h. The dried precipitate can be calcined in presence of air, at a temperature ranging from 300° C. to 700° C. for 2 h to 8 h.

Another aspect of the present invention concerns a crystalline cobalt (Co) molybdenum (Mo) catalyst. The crystalline CoMo catalyst can have a monoclinic crystalline structure. The crystalline CoMo catalyst can have an X-ray power diffraction pattern as substantially depicted in FIG. 2.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment discussed herein can be implemented with respect to any method or composition of the invention, and vice versa. Furthermore, compositions and systems of the invention can be used to achieve methods of the invention.

The following includes definitions of various terms and phrases used throughout this specification.

The term “monoclinic crystal structure” refers to a crystal that is described by three unequal-length vectors that form a rectangular prism with a parallelogram base, wherein two of said vectors are substantially perpendicular, while the third vector meets the other two at an angle other than 90°.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %,” “vol %,” or “mol.%” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%. “Essentially free” is defined as having no more than about 0.1% of a component.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Selectivity of a compound in a reaction is defined as: Selectivity of a compound one=(moles of compound one produced/total moles produced)*100

The process and systems of the present invention can “comprise,” “consist essentially of,” or “consist of” particular ingredients, components, compositions, steps, etc. disclosed throughout the specification. With respect to the transitional phrase “consisting essentially of,” in one non-limiting aspect, a basic and novel characteristic of the processes and the systems of the present invention are their abilities to produce methanol and ethanol from CO hydrogenation using crystalline cobalt molybdenum catalyst. The method can have a relatively high selectivity for methanol and ethanol (e.g., combined selectivity of methanol and ethanol of at least 50%).

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

FIG. 1: Thermal Gravimetric Analysis of the crystalline cobalt molybdenum catalyst.

FIG. 2: X-ray power diffraction of the crystalline cobalt molybdenum catalyst.

FIG. 3: Raman spectrum of the crystalline cobalt molybdenum catalyst.

FIG. 4: CO conversion percentage obtained with cobalt molybdenum catalyst.

### DETAILED DESCRIPTION OF THE INVENTION

A discovery has been made that provides a solution to low selectivity of methanol and ethanol obtained from CO hydrogenation. The solution is premised on using a crystalline cobalt molybdenum catalyst and hydrogenating CO using the catalyst. The combined selectivity of methanol and ethanol obtained from CO hydrogenation using the catalyst can be at least 50%, and combined selectivity of propanol and butanol obtained can be less than 20%.

These and other non-limiting aspects of the present invention are discussed in the following sections.

### A. Crystalline Cobalt Molybdenum Catalyst

In one aspect of the present invention, a crystalline cobalt molybdenum catalyst is described. The crystalline cobalt molybdenum catalyst can include a monoclinic crystalline structure. In some aspects, the monoclinic cobalt molybdenum catalyst can be a monoclinic cobalt molybdenum oxide. In some aspects, the monoclinic cobalt molybdenum oxide can be CoₓMo_{y}O_{z}, where x can be 0.5 to 1.5 or at least any one of, equal to any one of, or between any two of 0.5, 0.6, 0.7, 0.8, 0.9. 1, 1.1, 1.2, 1.3, 1.4 and 1.5, y can be 0.5 to 1.5 or at least any one of, equal to any one of, or between any two of 0.5, 0.6, 0.7, 0.8, 0.9. 1, 1.1, 1.2, 1.3, 1.4 and 1.5, and z can balance the valencies of Co and Mo. In certain aspects, z can be 3.5 to 4.5 or at least any one of, equal to any one of, or between any two of 3.5, 3.6, 3.7, 3.8, 3.9. 4, 4.1, 4.2, 4.3, 4.4 and 4.5. In some particular aspects, the monoclinic cobalt molybdenum oxide can include α-CoMoO₄ and β-CoMoO₄ at a α-CoMoO₄ to β-CoMoO₄ wt. % ratio 15:85 to 35:65 or at least any one of, equal to any one of, or between any two 15:85, 16:84, 17:83, 18:82, 19:81, 20:80, 21:79, 22:78, 23:77, 24:76, 25:75, 26:74, 27:73, 28:72, 29:71, 30:70, 31:69, 32:68, 33:67, 34:66 and 35:65. In some aspects, the catalyst can be a bulk catalyst and does not contain a support. In some aspects, the catalyst does not contain a cobalt sulfide, a molybdenum sulfide and/or a metal sulfide. In some aspects, the catalyst does not contain an alkali metal. In some aspects, the catalyst does not contain an alkaline earth metal. Monoclinic CoMoO₄ can exist as α-CoMoO₄ and β-CoMoO₄. Although the two forms have similar stoichiometry, the coordination of Mo is different between α-CoMoO₄ and β-CoMoO₄. Coordination of Mo is octahedral in α-CoMoO₄ whereas in β-CoMoO₄ Mo has tetrahedral coordination. It was surprisingly found that the product distribution obtained from CO hydrogenation with a monoclinic CoMoO₄ depends on the ratio of α-CoMoO₄ and β-CoMoO₄. At α-CoMoO₄ and β-CoMoO₄ wt. % ratio 15:85 to 35:65, methanol and ethanol can be obtained with high selectivity.

### B. Methods of Making a Crystalline Cobalt Molybdenum Catalyst

The crystalline cobalt molybdenum catalyst can be prepared via co-precipitation method. A cobalt compound and a molybdenum compound can be dissolved in two separate solutions. The solutions can be heated to dissolve the compounds. The solutions then can be mixed and a precipitate containing cobalt and molybdenum can be collected. The cobalt and molybdenum can be mixed at molar ratio of 0.5:1 to 1:0.5, preferably about 1:1. In some embodiments, a cobalt compound and a molybdenum can be added to a same solution and a precipitate can be collected from the solution. In some aspects, the solutions can be aqueous solutions. The cobalt compound can be any acceptable cobalt compound, non-limiting examples of which include cobalt acetate, cobalt acetyl acetonate, cobalt citrate, or a combination thereof. In some preferred instances, the cobalt compound is cobalt acetate. The molybdenum compound can be any acceptable molybdenum compound, non-limiting examples of which include ammonium heptamolybdate, molybidic acid, phosphomolybdic acid, potassium heptamolybdate, or a combination thereof. In some preferred instances, the molybdenum compound is ammonium heptamolybdate. The precipitate can be dried at a temperature 70° C. to 150° C. or at least any one of, equal to any one of, or between any two of 70° C., 80° C., 90° C., 100° C., 110° C., 120° C., 130° C., 140° C. and 150° C. for 3 h to 10 h or at least any one of, equal to any one of, or between any two of 3 h, 4 h, 5 h, 6 h, 7 h, 8 h and 10 h to obtain a dried precipitate. The dried precipitate can be calcined in air at a temperature 300° C. to 700° C. or at least any one of, equal to any one of, or between any two of 300° C., 400° C., 500° C., 600° C., and 700° C. for 2 h to 8 h or at least any one of, equal to any one of, or between any two of 2 h, 3 h, 4 h, 5 h, 6 h, 7 h and 8 h to obtain the crystalline cobalt molybdenum.

In some aspects, the crystalline cobalt molybdenum catalyst can be reduced to obtain an activated cobalt molybdenum catalyst. In some aspects, the activation process can include contacting a crystalline cobalt molybdenum catalyst with a stream containing H₂ at a temperature 200° C. to 500° C. or at least any one of, equal to any one of, or between any two of 200° C., 250° C., 300° C., 350° C., 400° C., 450° C. and 500° C., at a GHSV 1000 h⁻¹ to 3000 h⁻¹ or at least any one of, equal to any one of, or between any two of 1000 h⁻¹, 1100 h⁻¹, 1200 h⁻¹, 1300 h⁻¹, 1400 h⁻¹, 1500 h⁻¹, 1600 h⁻¹, 1700 h⁻¹, 1800 h⁻¹ , 1900 h⁻¹, 2000 h⁻¹, 2100 h⁻¹, 2200 h⁻¹, 2300 h⁻¹, 2400 h⁻¹, 2500 h⁻¹, 2600 h⁻¹, 2700 h⁻¹, 2800 h⁻¹, 2900 h⁻', and 3000 h⁻' and/or at a pressure 25 bar to 90 bar at least any one of, equal to any one of, or between any two of 25 bar, 30 bar, 40 bar, 50 bar, 60 bar, 70 bar, 80 bar and 90 bar for 8 h to 20 h or at least any one of, equal to any one of, or between any two of 8 h, 10 h 12 h, 14 h, 16 h, 18 h and 20 h to reduce and activate the catalyst.

### C. Methods of Using the Crystalline Cobalt Molybdenum Catalyst

The crystalline cobalt molybdenum catalysts of the present invention can be used to catalyze the hydrogenation of CO to produce C1 and C2 alcohols with relatively high selectivity. By way of example, a gaseous stream containing CO and H₂ can be contacted with a crystalline cobalt molybdenum catalyst of the present invention under conditions suitable to produce a products stream containing methanol and ethanol by CO hydrogenation. The conditions can include a pressure of 25 bar to 90 bar or at least any one of, equal to any one of, or between any two of 25 bar, 35 bar, 45 bar, 55 bar, 65 bar, 75 bar, 85 bar, and 90 bar, GHSV 1000 h⁻' to 3000 h⁻' or at least any one of, equal to any one of, or between any two of 1000 h⁻¹, 1500 h⁻¹, 2000 h⁻¹, 2500 h⁻¹ and 3000 h⁻¹ and/or a temperature 150° C. to 450° C. or at least any one of, equal to any one of, or between any two of 150° C., 200° C., 250° C., 300° C., 350° C., 400° C., and 450° C. The molar ratio of H₂ and CO in the gaseous stream can be 0.5:1 to 3:1 or at least any one of, equal to any one of, or between any two of 0.5:1, 0.8:1, 1:1, 1.2:1, 1.5:1, 2:1, 2.5:1, and 3:1. In some aspects, the ratio of CO and H₂ is about 1:1. In some aspects, the gaseous stream can contain an inert gas, such as nitrogen, in an amount ranging from 1 to 20%, In some aspects, the gaseous stream is a synthesis gas stream. In some aspects, the crystalline cobalt molybdenum catalyst can be activated prior to contacting the catalyst with the gaseous stream. In some aspects, the crystalline cobalt molybdenum catalyst can be positioned in a stationary bed of a reactor. The stream containing H₂ can be passed over or through the stationary bed to reduce and activate the catalyst and, the gaseous stream can be passed over or through the stationary bed to form methanol and ethanol.

In some aspects, the CO conversion can be 20% to 40% or at least any one of, equal to any one of, or between any two of 20%, 21%, 22%, 23%, 24%, 25%, 26%, 27%, 28%, 29%, 30%, 31%, 32%, 33%, 34%, 35%, 36%, 37%, 38%, 39% and 40%. In some aspects, the combined selectivity of the methanol and ethanol produced from the CO and H₂ can be 50% to 75% or at least any one of, equal to any one of, or between any two of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, and 75%. In some particular aspects, selectivity of the methanol can be 20% to 40% or at least any one of, equal to any one of, or between any two of 20%, 21%, 22%, 23%, 24%, 25%, 26%, 27%, 28%, 29%, 30%, 31%, 32%, 33%, 34%, 35%, 36%, 37%, 38%, 39% and 40%. In some particular aspects, selectivity of the ethanol can be 20% to 40% or at least any one of, equal to any one of, or between any two of 20%, 21%, 22%, 23%, 24%, 25%, 26%, 27%, 28%, 29%, 30%, 31%, 32%, 33%, 34%, 35%, 36%, 37%, 38%, 39% and 40%. In some aspects, combined selectivity of propanol and butanol if produced from the CO and H₂ can be less than 20%, preferably less than 15%, more preferably less than 10%.

In some particular aspects, selectivity of propanol if produced can be less than 10%, preferably less than 7%, more preferably less than 5%. In some particular aspects, selectivity of butanol if produced can be less than 10%, preferably less than 7%, more preferably less than 5%. In a further aspect, the ethanol produced can be dehydrated to obtain ethylene. Dehydration can be performed at a temperature above alcohol boiling point in the presence of suitable ethanol dehydration catalyst, such as an acid-type catalyst, e.g., cesium-doped silicotungstic acid supported on alumina.

### EXAMPLES

As part of the disclosure of the present invention, specific examples are included below. The examples are for illustrative purposes only and are not intended to limit the invention. Those of ordinary skill in the art will readily recognize parameters that can be changed or modified to yield essentially the same results.

### Example 1

### Crystalline Cobalt Molybdenum Catalyst Preparation and Activity Evaluation

Catalyst preparation. Catalysts were prepared via co-precipitation method.

Separate solutions of cobalt acetate (12.45 g, 100 ml d.H₂₀) and ammonium heptamolybdate (8.45 g, 100 ml d.H₂O) were heated to 65° C. to dissolve the salts. While under stirring and the molybdenum solution heated at 65° C., the cobalt solution was added dropwise using a separating funnel and aged for 2 h. The solution was then filtered without washing and the dark purple precipitate was dried in an oven (110° C.) for 6 h. The catalyst precursor was calcined (500° C., static air, 10° C./min, 4 h) resulting in the cobalt molybdenum catalyst.

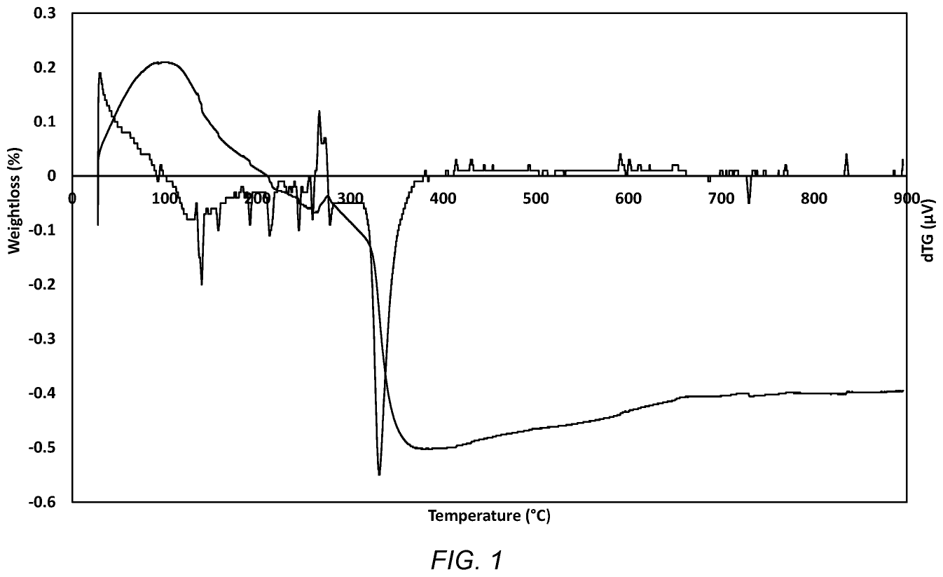

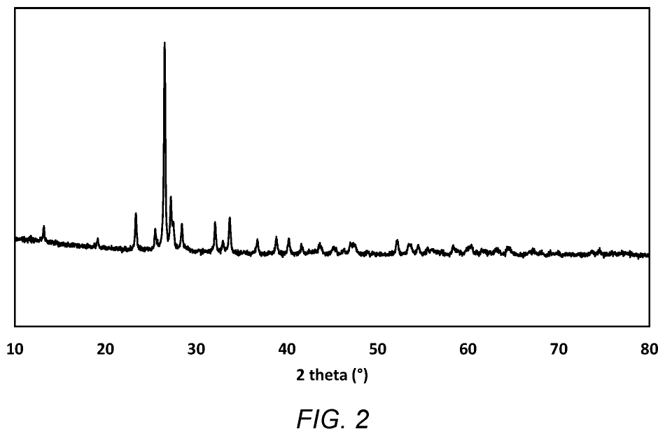

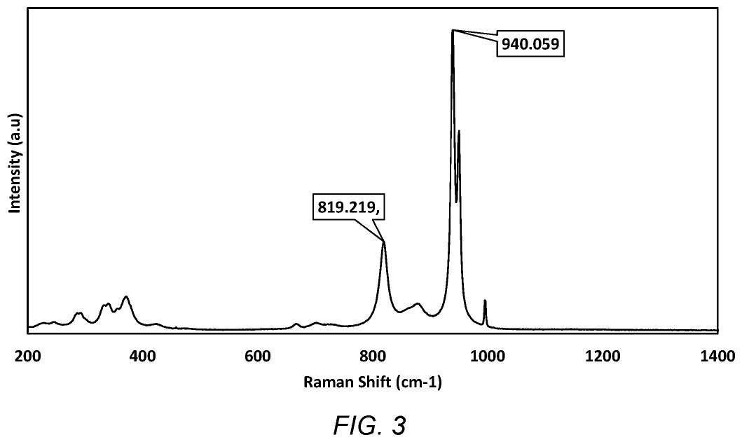

Catalyst Characterization. Catalyst characterization results are shown in FIG. 1-3. Thermogravimetric analysis (TGA) was used to assess the decomposition of the cobalt molybdate precursor to the final state catalyst (FIG. 1). TGA shows a weight loss from 300° C. to 400° C. showing the acetate evolving to CO₂ shown by the negative peak (exothermic). This confirms the calcination at 500° C. would result in decomposition of the acetates and nitrates. X-ray powder diffraction (FIG. 2) shows reflections corresponding to monoclinic CoMoO₄ with α-CoMoO₄ and β-CoMoO₄ at a wt. % ratio 25:75. The peak at 11.7°, which is the main reflection of molybdenum trioxide is also observed suggesting phase separation as 1:1 Co:Mo ratio was used. The excess can also be seen in the Raman spectrum 819 cm⁻¹ (FIG. 3).

Catalyst activity and selectivity evaluation. The catalysts were evaluated for the activity and selectivity calculations along with short term as well as long studies of the catalyst stabilities. Prior to activity measurement, the catalysts were subjected to activation procedure, by reducing the catalyst with a H₂ (H₂, 100 ml/min, 350° C., 1° C./min, 16 h). Catalytic evaluation was carried out in high throughput fixed bed flow reactor setup housed in temperature-controlled system fitted with regulators to maintain pressure during the reaction. The products of the reactions were analyzed through online GC analysis. The evaluation was carried out under the following conditions unless otherwise mentioned elsewhere; 47.5% H₂/47.5% CO/5% N₂, 75 Bar, 300° C., 1° C./min, 48 h stabilization, 100 ml/min, 50% SiC mix. The mass balance of the reactions is calculated to be 95±5%. Dehydration of alcohols produced can be carried out at a temperature above their boiling points and in the presence of and acid type catalyst for example cesium doped silicotungstic acid supported on alumina.

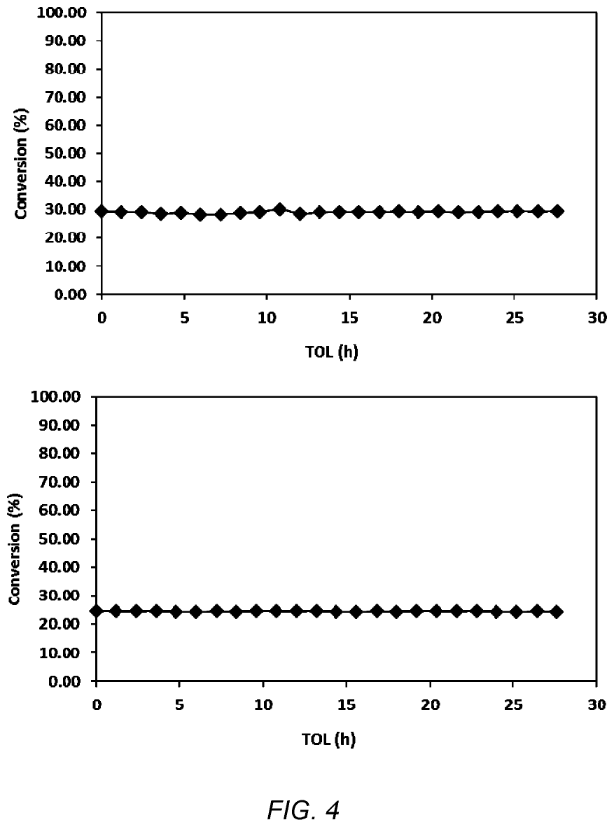

The results of catalyst testing are illustrated in FIG. 4 and Table 1. Duplicate sets of data show that the catalyst testing is reproducible. The catalyst is stable under reaction conditions, after a stabilization time of 48 h, with CO conversion at 24-30% (FIG. 4, Table 1). The catalyst produces substantial amount of MeOH and EtOH (ca. 60%) with small amount of propanol and butanol (ca. 10%) (Table 1).

TABLE 1
Product selectivity profile obtained from CO hydrogenation with the crystalline
cobalt molybdenum catalyst.
Conversion/Selectivity (mole %)

| TOS [h⁻¹] | Methanol | Ethanol | Propanol | Butanol | C_{2—}C₇ | CH₄ | CO₂ | Conversion |
|---|---|---|---|---|---|---|---|---|
| 0 | 25 | 25 | 8 | 7 | 19 | 0 | 15 | 30 |
| 2 | 33 | 28 | 7 | 6 | 10 | 0 | 16 | 30 |
| 4 | 25 | 25 | 9 | 8 | 19 | 0 | 14 | 30 |
| 5 | 32 | 30 | 8 | 7 | 10 | 0 | 13 | 30 |
| 6 | 30 | 34 | 8 | 7 | 8 | 0 | 13 | 30 |
| 7 | 30 | 28 | 9 | 9 | 10 | 0 | 14 | 30 |
| 8 | 29 | 29 | 9 | 8 | 10 | 0 | 15 | 30 |
| 10 | 31 | 30 | 9 | 9 | 9 | 0 | 12 | 30 |
| 11 | 28 | 31 | 6 | 6 | 18 | 0 | 11 | 30 |
| 12 | 33 | 26 | 8 | 7 | 11 | 0 | 15 | 30 |
| 13 | 30 | 30 | 7 | 7 | 10 | 0 | 16 | 30 |
| 14 | 30 | 29 | 8 | 7 | 15 | 0 | 11 | 30 |
| 16 | 29 | 35 | 7 | 7 | 10 | 0 | 12 | 30 |
| 17 | 34 | 30 | 6 | 5 | 10 | 0 | 15 | 30 |
| 18 | 28 | 27 | 6 | 6 | 20 | 0 | 13 | 30 |
| 19 | 30 | 29 | 10 | 9 | 10 | 0 | 12 | 30 |
| 20 | 28 | 35 | 7 | 7 | 8 | 0 | 15 | 30 |
| 22 | 30 | 27 | 4 | 4 | 20 | 0 | 15 | 30 |
| 23 | 28 | 27 | 8 | 7 | 13 | 0 | 17 | 30 |
| 24 | 30 | 29 | 8 | 8 | 10 | 0 | 15 | 30 |
| 25 | 29 | 33 | 3 | 3 | 18 | 0 | 14 | 30 |
| 26 | 33 | 30 | 5 | 4 | 12 | 0 | 16 | 30 |
| 28 | 30 | 29 | 7 | 7 | 10 | 0 | 17 | 30 |

In the context of the present invention, at least the following 20 embodiments are described. Embodiment 1 is a method for producing methanol and ethanol. The method includes contacting a gaseous stream containing carbon monoxide (CO) and hydrogen (Hz) with a crystalline cobalt molybdenum catalyst under conditions suitable to produce a products stream containing methanol and ethanol from the CO and Hz. Embodiment 2 is the method of embodiment 1, wherein the crystalline cobalt molybdenum catalyst contains a monoclinic crystalline structure. Embodiment 3 is the method of embodiment 2, wherein the crystalline cobalt molybdenum catalyst is a monoclinic cobalt molybdenum oxide. Embodiment 4 is the method of embodiment 3, wherein the monoclinic cobalt molybdenum oxide is CoₓMo_{y}O_{z}, wherein x ranges from 0.5 to 1.5, y ranges from 0.5 to 1.5, and z ranges from 3.5 to 4.5. Embodiment 5 is the method of embodiment 4, wherein the monoclinic cobalt molybdenum oxide contains α-CoMoO₄ and β-CoMoO₄ at a α-CoM004 to β-CoMoO₄ wt. % ratio 15:85 to 35:65. Embodiment 6 is the method of any one of embodiments 1 to 5, wherein the crystalline cobalt molybdenum catalyst is activated prior to contacting the catalyst with the gaseous stream. Embodiment 7 is the method of embodiment 6, wherein the crystalline cobalt molybdenum catalyst is activated by reducing the crystalline cobalt molybdenum catalyst, preferably with a stream containing hydrogen (H₂), and more preferably, at a temperature 200° C. to 500° C., at a GHSV of 1000 to 3000 , and at a pressure 25 bar to 90 bar. Embodiment 8 is the method of any one of embodiments 1 to 7, wherein the contacting conditions include a pressure of 25 bar to 90 bar, a GHSV of 1000 to 3000 and a temperature of 150° C. to 450° C. Embodiment 9 is the method of any one of embodiments 1 to 8, wherein the CO conversion is 20% to 40%. Embodiment 10 is the method of any one of embodiments 1 to 9, wherein the combined selectivity of the methanol and ethanol is 50% to 75%, and the combined selectivity of propanol and butanol is less than 20%. Embodiment 11 is the method of any one of embodiments 1 to 10, wherein the selectivity of methanol is 20% to 40%, the selectivity of ethanol is 20% to 40%, the selectivity of propanol is less than 10%, and the selectivity of butanol is less than 10%. Embodiment 12 is the method of any one of embodiments 1 to 11, wherein the molar ratio of H₂ and CO in the gaseous stream is 0.5:1 to 3:1. Embodiment 13 is the method of any one of embodiments 1 to 12, wherein the crystalline cobalt molybdenum catalyst is a bulk catalyst.

Embodiment 14 is a method for making a catalyst containing a crystalline cobalt molybdenum catalyst. The method includes preparing a solution containing a cobalt compound and a molybdenum compound and collecting a precipitate from the solution. The method also includes drying the precipitate to obtain a dried precipitate. The method further includes calcining the dried precipitate to obtain the crystalline cobalt molybdenum catalyst. Embodiment 15 is the method of embodiment 14, wherein the cobalt compound is cobalt acetate, cobalt acetyl acetonate, cobalt citrate, or a combination thereof, preferably cobalt acetate, and the molybdenum compound is ammonium heptamolybdate, molybidic acid, phosphomolybdic acid, potassium heptamolybdate or a combination thereof, preferably ammonium heptamolybdate. Embodiment 16 is the method of either one of embodiments 14 or 15, wherein the precipitate is dried at a temperature of 70° C. to 150° C. for 3 h to 10 h. Embodiment 17 is the method of any one of embodiments 14 to 16, wherein the dried precipitate is calcined in presence of air at a temperature ranging from 300° C. to 700° C. for 2 h to 8 h. Embodiment 18 is the method of any one of embodiments 14 to 17, wherein the crystalline cobalt molybdenum catalyst contains monoclinic cobalt molybdenum oxide. Embodiment 19 is the method of embodiment 18, wherein the monoclinic cobalt molybdenum oxide is CoₓMo_{y}O_{z}, wherein x ranges from 0.5 to 1.5, y ranges from 0.5 to 1.5, and z ranges from 3.5 to 4.5. Embodiment 20 is the method of embodiment 19, wherein the monoclinic cobalt molybdenum oxide contains α-CoMoO₄ and β-CoMoO₄ at a α-CoMoO₄ to β-CoMoO₄ wt. % ratio 15:85 to 35:65.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method for producing methanol and ethanol, the method comprising:
contacting a gaseous stream comprising carbon monoxide (CO) and hydrogen (H₂) with a crystalline cobalt molybdenum catalyst under conditions suitable to produce a products stream comprising methanol and ethanol from the CO and H₂.

2. The method of claim 1, wherein the crystalline cobalt molybdenum catalyst comprises a monoclinic crystalline structure.

3. The method of claim 2, wherein the crystalline cobalt molybdenum catalyst is a monoclinic cobalt molybdenum oxide.

4. The method of claim 3, wherein the monoclinic cobalt molybdenum oxide is CoₓMo_{y}O_{z},
wherein x ranges from 0.5 to 1.5, y ranges from 0.5 to 1.5, and z ranges from 3.5 to 4.5.

5. The method of claim 4, wherein the monoclinic cobalt molybdenum oxide comprises α-CoMoO₄ and β-CoMoO₄ at a α-CoMoO₄ to β-CoMoO₄ wt. % ratio 15:85 to 35:65.

6. The method of claim 1, wherein the crystalline cobalt molybdenum catalyst is activated prior to contacting the catalyst with the gaseous stream.

7. The method of claim 6, wherein the crystalline cobalt molybdenum catalyst is activated by reducing the crystalline cobalt molybdenum catalyst, preferably with a stream comprising hydrogen (H₂), and more preferably, at a temperature 200° C. to 500° C., at a GHSV of 1000 h⁻¹ to 3000 h⁻¹, and at a pressure 25 bar to 90 bar.

8. The method of claim 1, wherein the contacting conditions comprise a pressure of 25 bar to 90 bar, a GHSV of 1000 h⁻¹ to 3000 h⁻¹, and a temperature of 150° C. to 450° C.

9. The method of claim 1, wherein the CO conversion is 20% to 40%.

10. The method of claim 1, wherein the combined selectivity of the methanol and ethanol is 50% to 75%, and the combined selectivity of propanol and butanol is less than 20%.

11. The method of claim 1, wherein the selectivity of methanol is 20% to 40%, the selectivity of ethanol is 20% to 40%, the selectivity of propanol is less than 10%, and the selectivity of butanol is less than 10%.

12. The method of claim 1, wherein the molar ratio of H₂ and CO in the gaseous stream is 0.5:1 to 3:1.

13. The method of claim 1, wherein the crystalline cobalt molybdenum catalyst is a bulk catalyst.

14. A method for making a catalyst comprising a crystalline cobalt molybdenum catalyst, the method comprising:
preparing a solution comprising a cobalt compound and a molybdenum compound and collecting a precipitate from the solution;
drying the precipitate to obtain a dried precipitate; and
calcining the dried precipitate to obtain the crystalline cobalt molybdenum catalyst.

15. The method of claim 14, wherein the cobalt compound is cobalt acetate, cobalt acetyl acetonate, cobalt citrate, or a combination thereof, preferably cobalt acetate, and the molybdenum compound is ammonium heptamolybdate, molybidic acid, phosphomolybdic acid, potassium heptamolybdate or a combination thereof, preferably ammonium heptamolybdate.

16. The method of claim 14, wherein the precipitate is dried at a temperature of 70° C. to 150° C. for 3 h to 10 h.

17. The method of claim 14, wherein the dried precipitate is calcined in presence of air at a temperature ranging from 300° C. to 700° C. for 2 h to 8 h.

18. The method of claim 14, wherein the crystalline cobalt molybdenum catalyst comprises monoclinic cobalt molybdenum oxide.

19. The method of claim 18, wherein the monoclinic cobalt molybdenum oxide is CoₓMo_{y}O_{z},
wherein x ranges from 0.5 to 1.5, y ranges from 0.5 to 1.5, and z ranges from 3.5 to 4.5.

20. The method of claim 19, wherein the monoclinic cobalt molybdenum oxide comprises α-CoMoO₄ and β-CoMoO₄ at a α-CoMoO₄ to β-CoMoO₄ wt. % ratio 15:85 to 35:65.
